(19) United States
(12) Patent Application Publication
(10) Pub. No.: US 2022/0104982 A1
(43) Pub. Date: Apr. 7, 2022

(54) PORTABLE NEGATIVE PRESSURE ISOLATION UNIT

(71) Applicant: MACH32 INC., Edmonton (CA)

(72) Inventors: Marc CURIAL, Edmonton (CA); Chris TERRIFF, Edmonton (CA); Will COMEAU, Edmonton (CA); Ryley MCCONKEY, Waterloo (CA)

(21) Appl. No.: 17/490,880

(22) Filed: Sep. 30, 2021

Related U.S. Application Data
(60) Provisional application No. 63/086,301, filed on Oct. 1, 2020.

(51) Int. Cl. A61G 10/00; A61G 10/02

(52) U.S. Cl. CPC A61G 10/005; A61B 46/20; A61G 10/023

(57) ABSTRACT

The present disclosure provides an isolation unit to protect uninfected persons from a patient infected with a respiratory disease during a use case. The isolation unit generally includes a frame and cover configured to form an enclosed space surrounding the head, neck and shoulders of the patient, the enclosed space containing air and a fan/filter system in fluid communication with the enclosed space and which includes a vacuum to create a negative pressure within the enclosed space to withdraw air in the enclosed space to the fan/filter system and a filter to remove any contaminants in the air.

### CROSS-REFERENCE TO RELATED APPLICATION

This application claims the benefit of U.S. Provisional Patent Application Ser. No. 63/086,301 filed on Oct. 1, 2020. The content of the aforementioned application is incorporated herein by reference.

### FIELD

The present disclosure generally relates to a medical device, and more particularly to an isolation unit capable of creating a negative pressure environment within a temporary enclosed space. The isolation unit may be used to enclose and isolate the space and air contained therein around the upper body and head of a patient (for example, a patient infected with a respiratory disease), withdraw the air and any airborne and aerosolized viral particles that may have originated from the patient within the enclosed space and remove such particles from the air prior to expelling the air to the surrounding environment.

### BACKGROUND

Aerosol generating medical procedures (AGMP's), such as intubation, CPAP, BiPAP, and high flow oxygen therapy pose a significant threat to healthcare workers during the COVID-19 pandemic. These procedures produce aerosolized viral particles, which dramatically increase the risk of infection for any hospital staff in the room while the procedure is taking place. Due to the high-risk nature of these procedures, they are now banned or performed in a severely limited capacity in hospitals world-wide. Often these procedures are performed in a modified capacity designed to reduce staff exposure and these modified procedures carry a higher patient mortality rate than their full-capacity counterparts while still carrying significant risk to any caregiver that performs them in this limited fashion. Unfortunately, AGMP's are necessary for severe COVID-19 patients who often develop Acute Respiratory Distress Syndrome (ARDS). ARDS patients cannot ventilate without mechanical assistance, and getting that assistance requires hospital staff to undergo an AGMP.

To protect healthcare workers during AGMP's, several groups have developed products, colloquially known as intubation hoods or intubation boxes, which act as a barrier between hospital staff and the patient. Intubation boxes function on the level of personal protective equipment, which is the least effective method of hazard control as defined by the Occupational Safety and Health Administration (OSHA). These hoods are almost invariably made from clear polycarbonate panels with cut-outs for patient access. Relying on proper sealing to minimize the loss of aerosolized viral particles from the spaces they enclose, intubation boxes feature no system to remove or filter the contaminated air. In essence, these devices function as a splatter guard against large droplets only and do not contain aerosolized particles. Intubation hoods may actually increase the risk of infection for healthcare staff through a false sense of security. Additionally, some of these devices have been postulated to focus the generated aerosols through openings and towards medical staff in a concentrated stream. Hospital boards may incorrectly assume that these hoods offer patient isolation and may begin to permit their staff to perform AGMP's in a less limited or full capacity because of this assumption. Indeed, several of these hoods are marketed using statements like “granting safe access to patient airways” and “keeping both parties safe from germs and contaminants spread through medical procedures.” As these hoods are not perfectly sealed and offer no method of removing aerosolized viral particles from the space they enclose, they carry a significant risk of contamination to the healthcare worker through leakage. Thus, performing any AGMP in a less limited capacity than currently allowed for while using an intubation hood will likely increase, not decrease, the risk of hospital staff contracting COVID-19. Most other strategies to mitigate risks are non-disposable, non-adjustable, neutral pressure, and have a limited scope.

Examples of known systems capable of isolating a patient include, for example, the systems described in:
CN Pat. Nos. 2,631,473 and 2,712,320 which disclose negative pressure isolating hospital beds;
CN Pat. Nos. 2,841,852 and 201,263,764 which disclose inflatable negative pressure isolating units;
CN Pat No. 2,613,280 which discloses negative pressure type isolating clothing; and
U.S. Pat No. 7,479,103 which discloses a portable isolation enclosure about a bed with a filtration system;
   It is desirable to improve upon these state-of-the-art systems by providing a new system that is portable, stable on standard hospital beds and operating room tables, capable of generating negative pressure sufficient to capture and contain aerosols originating from a patient inside of a small, temporary enclosed space and remove such aerosols from the air contained within the enclosed space before the air is expelled to the surrounding environment.

### SUMMARY

The present disclosure provides a portable isolation unit designed to generate negative pressure inside a small, enclosed space surrounding a patient's head, neck, and shoulders via a combined fan/high-efficiency filter system, the enclosed space and fan/filter system each connected to a duct or similar air pathway so that the fan/filter system is in fluid communication with the enclosed space and the air contained therein. The isolation unit is designed specifically to contain airborne and aerosolized viral particles originating from the patient that are in the air within the enclosed space and the fan/filter system is designed to remove such viral particles before expelling the air to the surrounding environment. Thus, the isolation unit allows various procedures to take place on the patient while it is in use, and multiple healthcare providers can perform these procedures while being protected from any exhaled droplets and aerosols from the patient.

The isolation unit generally includes: a frame and an open-bottomed cover which fits around the frame, the frame and cover forming an enclosed space when placed on the patient to surround the patient's head, neck and shoulders; a duct; a duct connection system; and, a fan/filter system in fluid communication with the enclosed space and configured to: create a negative pressure within the enclosed space to withdraw air contained in enclosed space and into the fan/filter system; remove aerosols or droplets originating from the patient that are contained in the withdrawn air to form clean air; and expel the clean air to the surrounding environment.

### BRIEF DESCRIPTION OF THE DRAWINGS

FIG. 1 is a perspective view of an isolation unit according to an embodiment of the present disclosure;

FIG. 2 is an exploded view of the isolation unit of FIG. 1;

FIGS. 3 and 4 depict a frame and cover of the isolation unit of FIG. 1 forming an enclosed space around the head, neck and shoulders of a patient;

FIG. 5 is an exploded view of the duct and duct connector system of the isolation unit of FIG. 1;

FIG. 6 depicts a fan/filter system of the isolation unit of FIG. 1 supported by wheels and attached to the duct; and

FIG. 7 depicts a connection system of the isolation unit of FIG. 1 securing the frame to the cover.

### DETAILED DESCRIPTION

The following terms shall have the following meanings:

The term “comprising” and derivatives thereof are not intended to exclude the presence of any additional component, step or procedure, whether or not the same is disclosed herein. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

The articles “a” and “an” are used herein to refer to one or to more than one (i.e. to at least one) of the grammatical objects of the article. The phrases “in one embodiment”, “according to one embodiment” and the like generally mean the particular feature, structure, or characteristic following the phrase is included in at least one embodiment of the present disclosure, and may be included in more than one embodiment of the present disclosure. Importantly, such phrases do not necessarily refer to the same aspect. If the specification states a component or feature “may”, “can”, “could”, or “might” be included or have a characteristic, that particular component or feature is not required to be included or have the characteristic.

The term “duct” is used herein to refer to any enclosed pathway suitable for transferring air or other gases, including, but not limited to, flexible ductwork, hard ductwork, flexible tubing, hard tubing, or pipe.

The term “enclosed space” refers to a contained space formed by frame 1 and cover 2 and which can surround the head, neck and shoulders of a patient during use of the isolation unit and therefore contains the air within the contained space and any aerosols or droplets which have originated or originate from the patient's respiratory system.

The term “proximal” refers to the side closest to the patient's head while the term “distal” refers to the side closest to the patient's shoulders or torso.

The term “use case” refers to any situation in which the isolation unit may potentially be used, such as, but not limited to, intubation (both using a laryngoscope and a GlideScope), extubation, tracheostomy, and even some cases of user error such as when the enclosure is partially lifted off of the bed (to an approximate height of 1″).

The term “portable” refers to a device, machine or the like that can be relatively easily moved from one position to another. This is opposed to a device, machine or the like that is fixed or secured to a stationary object or that weighs enough that it requires a number of people to move it from one place to another.

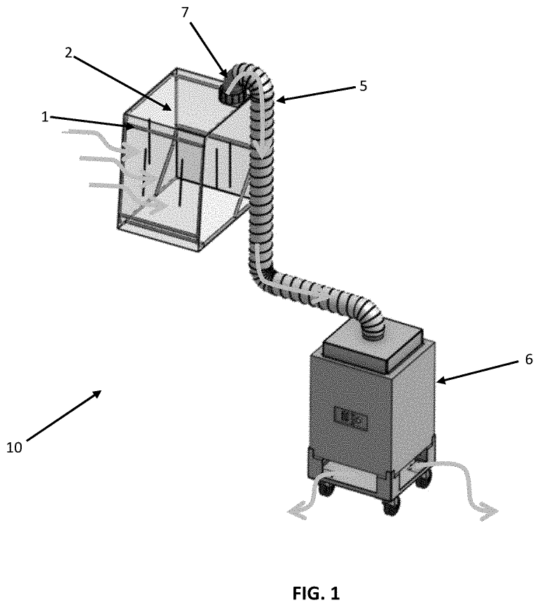

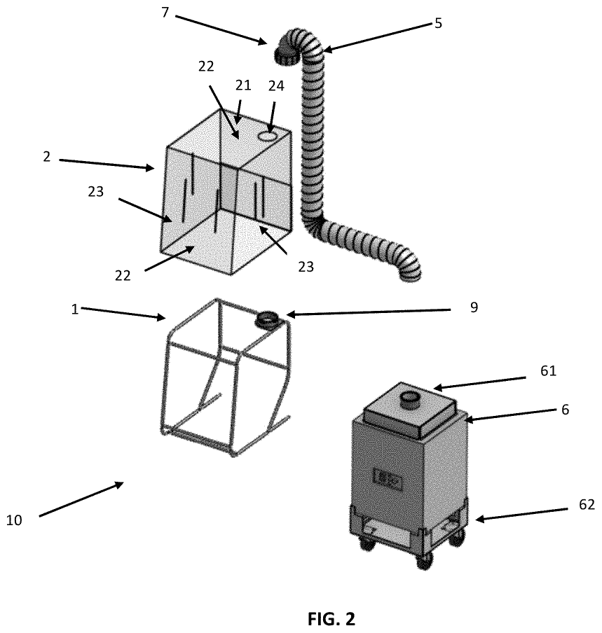

With reference to FIGS. 1 and 2, an isolation unit according to one embodiment is shown and generally designated by reference numeral 10. In one particular embodiment, the isolation unit 10 is portable. As shown in FIGS. 1 and 2, isolation unit 10 includes a frame 1 which may be surrounded by an open-bottomed cover 2 to form an enclosed space. In one embodiment, cover 2 may be connected to frame 1 via any suitable connection system, such as clips, Velcro, a screw fitting, a magnetic fitting, etc. The cover 2 and frame 1 are both sized and configured to be placed and fit around a patient's head, neck and shoulders. The isolation unit 10 further includes a duct 5 having opposing ends that in one embodiment, may be connected at one of its ends to cover 2 by a duct connection system 7 so as to permit air flow between the enclosed space and duct 5. The isolation unit 10 further includes a fan/filter system 6 which may be connected to the other end of the duct 5, the fan/filter system configured and designed to impart a negative pressure within the enclosed space and to draw air from within the enclosed space into the fan/filter system 6. Accordingly, the enclosed space formed by frame 1 and cover 2 is in fluid communication with fan/filter system 6 via duct 5. The fan/filter system 6 is also configured to filter the withdrawn air prior to expelling the air to the surrounding environment.

According to one embodiment, the isolation unit 10 is configured to operate in a negative isolation mode and is used to protect uninfected persons in the immediate vicinity from a patient infected with a respiratory disease. In particular, as shown in FIG. 1, the surrounding air is shown as entering the isolation unit 10, passing through the enclosed space where it may contact any aerosols or droplets originating from the patient, passing through duct 5 into the fan/filter system 6 where it is filtered and expelled back into the surrounding environment.

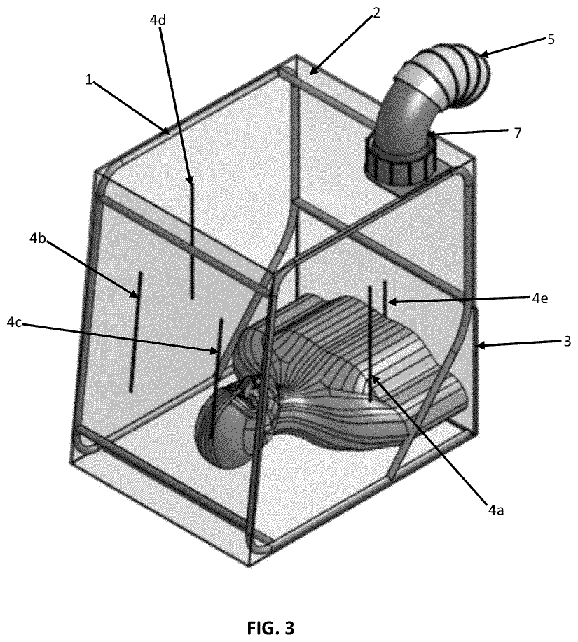

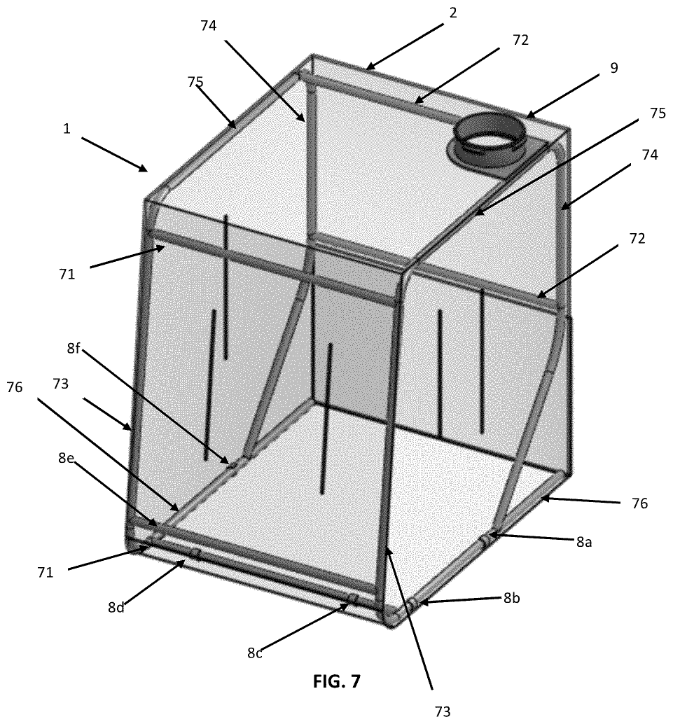

With reference now to FIGS. 1, 2, 3 and 7 the frame 1 is generally square or rectangularly shaped and includes upper and lower horizontal stabilizer bars 71 positioned at the proximal end of frame 1, upper and lower horizontal stabilizer bars 72 positioned at the distal end of frame 1, two spaced vertical sidebars 73 positioned at the proximal end of frame 1, two spaced vertical sidebars 74 positioned at the distal end of frame 1, two spaced upper connecting bars 75 connected to the upper proximal and upper distal horizontal stabilizer bars and two spaced lower connecting bars 76 connected to the proximal lower horizontal stabilizer bar and the lower ends of the two spaced vertical sidebars positioned at the distal end of frame 1, the lower connecting bars extending past such vertical sidebars. In other embodiments, there may be one or more additional upper and lower horizontal stabilizer bars, spaced vertical sidebars, or upper and lower connecting bars positioned to further stabilize the frame. As shown in FIGS. 2 and 7, the lower horizontal stabilizer bar positioned at the distal end of frame 1 is attached to the two spaced distal vertical sidebars, while the lower connecting bars extend past the lower ends of these vertical sidebars to form a taper that simplifies the removal or attachment of cover 2 to the frame 1 for the purpose of reducing set-up and take-down times. The frame 1 further includes a connector 9 (shown as a male connector) positioned at the distal end of frame 1 and attached to an upper connecting bar 75. In addition, the lower connecting bars 76 and lower horizontal stabilizer bar 71 positioned at the proximal end of frame 1 are configured to lay flush on a flat surface, such as a standard hospital bed, stretcher or operating table, for stabilization purposes. The frame 1 may be made of any rigid material such as plastic, composite, metal, steel, stainless steel, aluminum, etc. and is preferably lightweight. Furthermore, the frame 1 is designed to be reusable and easy to clean between uses. Finally, the frame 1 is sized to fit around a patient's head, neck and shoulders when placed on the hospital bed, stretcher or operating room table and may be configured to adjust to different patient chest sizes.

With continued reference to FIGS. 1, 2, 3 and 7, the open-bottomed cover 2 is generally square or rectangularly shaped and is configured to fit securely over the frame 1 to form an enclosed space. The cover 2 can be formed as a unitary piece and may be made of any impermeable material such as PVC, polyethylene, plastic, glass, etc. The cover 2 may also be transparent to allow for optimal visual clarity. Furthermore, the cover 2 may be designed for single-use to prevent contamination and eliminate cleaning time between uses, or it may be reusable. In one embodiment, the cover 2 includes an upper wall 21, proximal and distal walls 22, two spaced side walls 23 and an opened bottom. The upper wall 21 includes an orifice 24 positioned at its distal end. The orifice 24 is sized and configured to slidably fit over the connector 9 of frame 1 when the cover 2 is placed over frame 1. The cover 2 further includes access ports 4a, 4b, 4c, 4d and 4e located on the proximal and distal walls 22 and side walls 23 and are shaped and configured to allow for a healthcare provider or other to access the patient. Each access port is configured to minimize pressure loss from the isolation unit 10 in order to maintain the negative pressure inside the enclosed space so as to prevent external exposure to aerosols or airborne particles within the enclosed space regardless of whether the access ports are open when being accessed or are closed when not being accessed. Thus, the access ports allow for real-time access, and manipulation of the patient while providing protection from exhaled droplets and aerosols originating from the patient. In some embodiments, the access ports are positioned on the walls of cover 2 to allow healthcare providers space and multiple points of access in order to be capable of contacting the patient in a variety of use cases. In one embodiment, each access port may be a vertical or horizontal slit, although other known sizes/shapes which are configured to allow access to the patient may be used.

The cover 2 further includes drape 3 attached to the distal wall 22. The drape 3 is sized and configured to be moveable and to seal the enclosed space from the outside environment.

With reference to FIGS. 1, 2 and 5, the isolation unit 10 further includes duct 5, the duct having a front end and a back end. The duct 5 may be disposable or may be reusable and easy to clean and may be fitted with a cap system at one or both ends to prevent particulates from exiting the duct when not in use.

With continued reference to FIGS. 1, 2 and 5, the isolation unit 10 further includes a duct connection system 7. The duct connection system 7 is configured to securely connect the front end of duct 5 to the connector 9 of frame 1. In one embodiment, the duct connection 7 includes a female swivel 7a and shank 7b which are configured to securely fasten to a male threaded connector 9 of frame 1 to form an air-tight seal. In other embodiments, the coupling between the connector 9 of frame 1 and duct connection system 7 may be a threaded fitting, press fit, or any other secure connection mechanism which can form an air-tight seal.

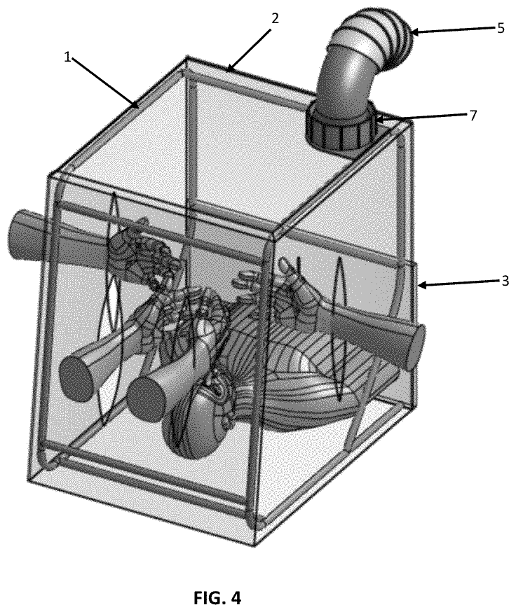

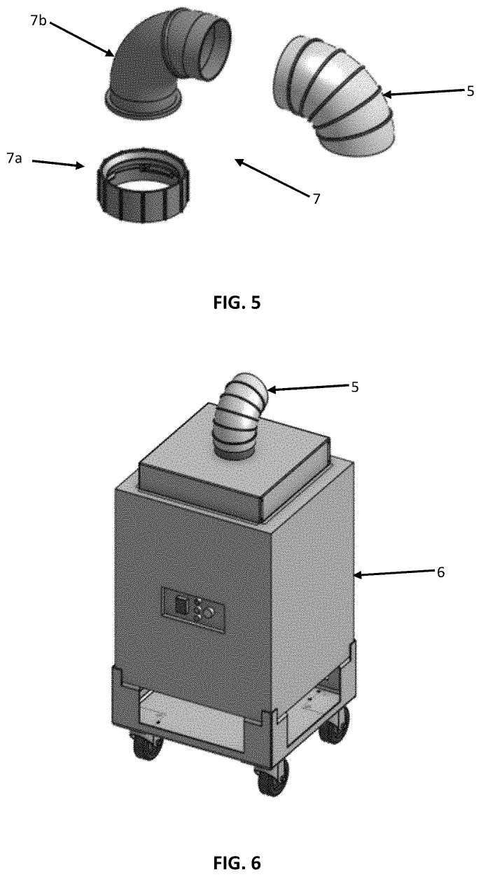

With reference now to FIGS. 1, 2 and 6, the isolation unit 10 further includes a fan/filter system 6. The fan/filter system 6 includes an inlet 61 configured and sized to attach to the back end of duct 5 and an outlet 62 configured to expel air that has been withdrawn from the enclosed space through duct 5 and into the fan/filter system to the surrounding environment. As shown in FIG. 6, the inlet 61 of the fan/filter system 6 is attached to the back end of duct 5 and the front end of duct 5 is attached to the duct connection system 7 shown in FIG. 5 to allow for fluid communication between the enclosed space and fan/filter system 6. Thus, the fan/filter system 6 is configured to provide continuous air changes in the enclosed space, wherein the contaminated air from inside the enclosed space will move through the duct 5 to the fan/filter system 6 where it can be filtered and expelled to the surrounding environment. The duct 5 is configured to allow the back end of duct 5 to attach and detach easily from the outlet 62 of the fan/filter system 6 to allow for easier transportation of the fan/filter system 6.

In one embodiment, the fan/filter system 6 includes a vacuum motor (not shown) configured to create high flow negative pressure inside of the enclosed space and to withdraw air from within the enclosed space. The fan/filter system 6 may also include a microbial and/or bacterial filter (for e.g. a HEPA filter, not shown) which is well known in the art and readily available from a large number of suppliers of products for the medical field and which is sized and configured to remove contaminants from the withdrawn air to form clean air before it is expelled through the outlet of the fan/filter system 6 to the surrounding environment. In one embodiment, the fan/filter system 6 is configured to produce at least a −2.5 pressure differential or at least a CSA guideline of −7.5 Pa pressure differential in the enclosed space relative to the outside space to create patient isolation in a variety of use cases for the purpose of protecting healthcare providers from harmful aerosolized or airborne viral particles originating from the patient. In such an embodiment, the fan/filter system 6 provides an air change rate of 2000 air changes per hour, thus changing the air within the enclosed space more than a Class 1 cleanroom. The fan/filter system 6 is also configured to reduce interference with laminar air flows in the room where the isolation unit 10 has been placed, and may include a plurality of outlets having large openings to minimize the velocity of cleaned air as it exits the fan/filter system 6. In still another embodiment, the fan/filter system 6 includes wheels for easy transportation and portability. The fan/filter system 6 may be made of any suitable material so as to be re-usable, as well as long-lasting. The fan/filter system 6 may also be configured to minimize noise emitted from the system to reduce noise disturbance to healthcare providers in the surrounding environment.

The fan/filter system 6 also includes an electrical connection which may be plugged into any standard 120V wall outlet, or into a battery system to turn the fan/filter system 6 on and activate the vacuum motor, allowing the isolation unit 10 to be operable in nearly any location.

With reference to FIG. 7, the isolation unit 10 may further include a connection system configured to securely fasten the frame 1 to cover 2. In one embodiment the connection system includes clips 8a, 8b, 8c, 8d, 8e, and 8f which may be used to create a secure connection between the frame 1 and cover 2 to prevent movement of the cover 2 so as to not disturb healthcare providers during any use cases. The clips may also be configured to offer further protection to healthcare providers by forming a tight seal between the frame 1 and cover 2 to reduce exposure from air inside the enclosed space.

The isolation unit 10 can generally be operated by placing the frame 1 and open-bottomed cover 2 over the patient's head, neck and shoulders or torso to form an enclosed space around the patient's respiratory system, sealing the distal end of the cover 2 by closing drape 3, connecting the front end of duct 5 to the connector of the frame 1 via the duct connection system 7, connecting the back end of duct 5 to the inlet of fan/filter system 6, turning the fan/filter system 6 on to create a negative pressure inside the enclosed space thereby withdrawing air contained within the enclosed space through the duct 5 and inlet of the fan/filter system 6, passing the air through a filter placed within the fan/filter system 6 and expelling the air through the outlet of the fan/filter system 6 to the surrounding environment.

Thus, in yet another embodiment there is provided a method for operating the isolation unit in a negative isolation mode to protect uninfected persons from a patient infected with a respiratory disease including: (i) placing the frame 1 and open-bottomed cover 2 of the isolation unit 10 over the head, neck and shoulders of the patient to form an enclosed space around the head, neck and shoulders of the patient, (ii) attaching the front end of the duct 5 to the connector 9 of the frame 1 via duct connection system 7, (iii) connecting the back end of duct 5 to the inlet of the fan/filter system 6, (iv) turning the fan/filter system 6 on to activate the vacuum motor of the fan/filter system 6 to create a negative pressure within the enclosed space to withdraw contaminated air contained within the enclosed space such that it passes through the duct 5 and into the inlet of the fan/filter system 6, the contaminated air including contaminants such as virus-contaminated aerosols or droplets originating from the patient, (v) passing the contaminated air through a filter placed within the fan/filter system 6 to remove the contaminants from the contaminated air to form clean air, and (vi) expelling the clean air through the outlet of the fan/filter system 6 and into the surrounding environment.

While making and using various embodiments of the present invention have been described in detail above, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

What is claimed is:

1. An isolation unit for a patient comprising:
(i) a frame configured to stably rest on a flat surface and sized to surround a head, neck and shoulders of the patient when placed around the patient and comprising a connector;
(ii) an open-bottomed cover configured to fit securely over the frame to form an enclosed space surrounding the patient's head, neck and shoulders and comprising an orifice that is sized and configured to slidably fit around the connector of the frame and at least one access port shaped and configured to allow access to the patient;
(iii) a duct having a front end and a back end;
(iv) a duct connection system to secure the connector of the frame to the front end of the duct;
(v) a fan/filter system comprising an inlet configured to connect to the back end of the duct, a vacuum motor configured to create a negative pressure within the enclosed space to withdraw air contained within the enclosed space and into the inlet, the air comprising viral contaminants, a filter configured to remove the viral contaminants from the air to form clean air, and an outlet configured to expel the clean air to the surrounding environment
wherein the isolation unit is operable to maintain a minimum pressure differential of −2.5 Pa during any use case.

2. The isolation unit of claim 1, wherein the isolation unit is portable.

3. The isolation unit of claim 2, further comprising a connection system configured to fasten the cover to the frame.

4. The isolation unit of claim 1, wherein the isolation unit is operable to maintain a minimum pressure differential of −7.5 Pa during any use case.

5. The isolation unit of claim 1, wherein the fan/filter system provides an air change rate of 2000 air changes per hour.

6. The isolation unit of claim 1, wherein the frame is made of a rigid material comprising plastic, composite, metal, steel, stainless steel or aluminum.

7. The isolation unit of claim 1, wherein the cover is transparent.

8. The isolation unit of claim 1, wherein the access port is a vertical or horizontal slit.

9. The isolation unit of claim 1, wherein the cover further comprises a drape.

10. A method of operating the isolation unit of claim 1 including: (i) placing the frame and open-bottomed cover over the head, neck and shoulders of the patient to form an enclosed space surrounding the head, neck and shoulders of the patient, (ii) attaching the front end of the duct to the connector of the frame via the duct connection system, (iii) connecting the back end of the duct to the inlet of the fan/filter system, (iv) turning the fan/filter system on to activate the vacuum motor and create a negative pressure within the enclosed space thereby withdrawing air contained within the enclosed space through the duct and into the inlet of the fan/filter system, wherein the air contains comprising contaminants including viral-contaminated aerosols or droplets originating from the patient, (v) passing the air through the filter to remove the contaminants from the air to form clean air, and (vi) expelling the clean air through the outlet of the fan/filter system and into the surrounding environment.
